(19) United States
(12) Patent Application Publication
(10) Pub. No.: US 2010/0049151 A1
(43) Pub. Date: Feb. 25, 2010

(54) Device for Treating Wounds

(75) Inventor: Martin Aicher, Zurich (CH)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: MEDELA HOLDING AG, Baar (CH)

(21) Appl. No.: 12/514,451

(22) PCT Filed: Nov. 9, 2007
(86) PCT No.: PCT/CH2007/000552

§ 371 Date: May 11, 2009

(30) Foreign Application Priority Data
Nov. 30, 2006 (CH) 1944/06

(51) Int. Cl. A61F 13/02; A61M 27/00; A61M 1/00

(52) U.S. Cl. 604/319; 602/43; 604/543

(57) ABSTRACT

The invention relates to a device for treating wounds of a patient by means of low pressure. Said device comprises a wound dressing and a cover film. Said cover film is placed on the wound dressing and is connected thereto. Said cover film is at least partially folded or rolled together before use. Said device can be easily adapted to the size and shape of the wound and is easy to handle.

### TECHNICAL FIELD

The invention relates to a device for treating wounds according to the preamble of claim 1.

### PRIOR ART

Open wounds that are too large or too badly inflamed to heal independently have long been a problem in medicine. It has been found that wound drainage using low pressure stimulates, supports and accelerates the healing of the wound.

This is described in U.S. Pat. No. 5,636,643, for example. The latter discloses a wound treatment device with a fluid-impermeable and gas-impermeable rigid cover that is placed over a wound and is secured to the healthy skin outside the margins of the wound. Underneath the cover, a wound dressing in the form of a foam is placed onto or into the wound. A negative pressure is generated in the cover from outside by means of a vacuum pump in order to accelerate the healing of the wound.

WO 03/018098 also describes a device for treating wounds, with a cover and with a porous pad that is placed onto the wound underneath the cover. This document proposes automated oscillation of the negative pressure in order to stimulate the healing of the wound.

WO 2006/056408 proposes that the cover be provided with supply devices for treatment substances. These treatment substances are removed together with the wound secretions through a drainage device.

WO 2006/048246 discloses a multicomponent dressing for wound treatment by means of negative pressure. This dressing comprises superabsorbent polymers, wherein the absorbed wound secretions remain bound to polymers in the wound cavity.

### DISCLOSURE OF THE INVENTION

It is therefore an object of the invention to create a device for treating wounds that can be used for different sizes of wounds.

This object is achieved by a device for treating wounds that has the features of claim 1.

The device according to the invention for treating wounds of a patient by means of low pressure comprises a wound dressing and a cover film or sheet, wherein the cover film or sheet is arranged on the wound dressing and connected thereto. The cover film is at least partially folded or rolled up before use.

In this way, the wound dressing can be given a shape adapted to the wound that is to be treated, for example by cutting it to shape, breaking it or tearing it, and can then be applied immediately to the wound. Since the covering and securing film is already connected fixedly to the wound dressing as an integral part thereof, handling is made very easy and causes the patient less pain.

Before use, a part of the cover film preferably lies flat on the wound dressing. This is preferably the part that surrounds the drainage tube. This has the advantage that the cover film can be unfolded or rolled out starting from a clearly defined location.

Before use, the cover film preferably has a smaller surface area than the wound dressing. In this way, it is only the wound dressing, not the cover film, that has to be adapted to the size of the wound. Should it nevertheless prove necessary to shorten the cover film, this can be done at a later stage, for example after the wound dressing has been introduced into or placed onto the wound.

The cover film preferably has a shape memory and can change from a basic shape to a shape for use. The wound is contracted in this way.

The device according to the invention thus combines filling of the wound bed, sealing of the wound and drainage in a very simple manner.

Other advantageous embodiments are set forth in the dependent claims.

### BRIEF DESCRIPTION OF THE DRAWINGS

The subject matter of the invention is explained below on the basis of a preferred illustrative embodiment depicted in the attached drawings, in which:

FIG. 1 shows a cross section through a device according to the invention, in a basic shape prior to its use, and

FIG. 2 shows a cross section through the device according to FIG. 1 during its use.

### WAYS TO CARRYING OUT THE INVENTION

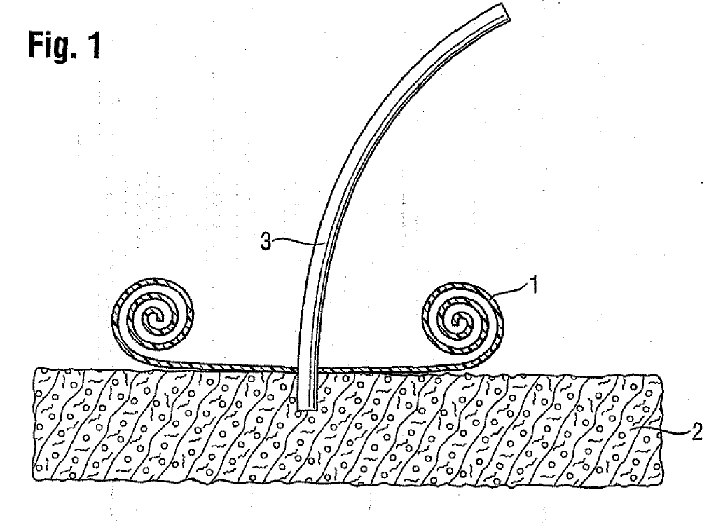

FIG. 1 shows a device according to the invention for treating wounds by means of low pressure, i.e. for wound drainage.

The device comprises a cover film 1, a wound dressing 2 and at least one tube, here a drainage tube 3. The drainage tube 3 extends through the film 1 and protrudes into the wound dressing 2.

The cover film 1 is connected fixedly to the wound dressing 2 or is an integral component part thereof. It can, for example, be bonded on adhesively, welded to it or connected to it with a form fit.

Before use, the cover film 1 is at least partially folded or rolled up. In FIG. 1, the cover film 1 is rolled up. The width of the cover film 1 is preferably adapted to the width of the wound. A part of the film 1, however, lies flat on the wound dressing 2 even before use. It is preferably the part of the cover film 1 surrounding the drainage tube 3.

The cover film 1 can be rectangular, in particular square, round or oval or can have another shape. It preferably has a shape that corresponds to the surface of the wound dressing 2, and it has a greater surface area than this. It should be preferably secured on the wound dressing 2 such that it protrudes past the latter about the entire circumference to a sufficient extent to serve as a securing film.

Before use, however, the cover film 1 preferably has a smaller surface area than the wound dressing 2, as can be seen from FIG. 1.

At least part of the cover film 1 is preferably self-adhesive such that, when unfolded or rolled out, it can be secured on the patient's healthy skin surrounding the wound. The part is preferably the entire peripheral area of the cover film 1. However, the whole surface of the cover film 1 directed toward the wound dressing 2 can be self-adhesive such that it adheres immediately to the wound dressing 2 when unfolded or rolled out. However, the cover film 2 can also be non-self-adhesive and can be secured on the skin by an additional adhesive means, for example an adhesive strip.

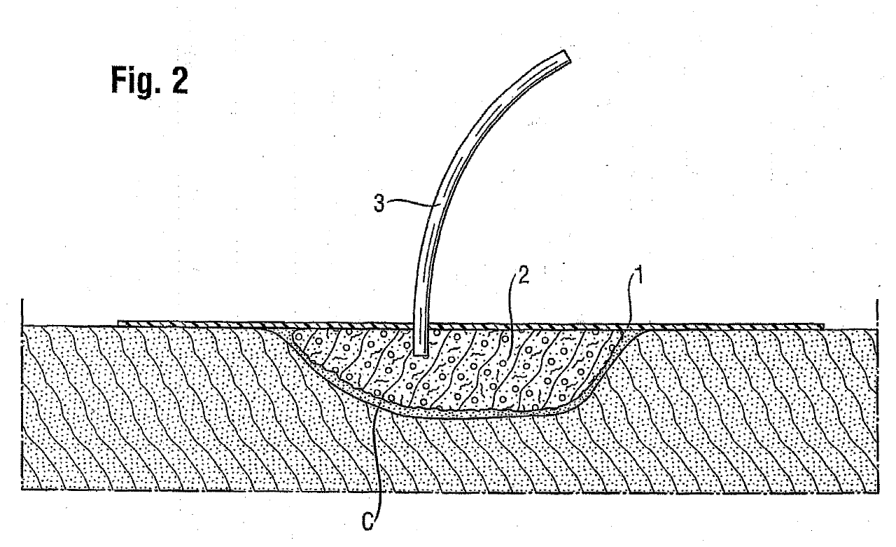

The cover film 1 and the wound dressing 2 thus respectively cover and fill the cavity C of the wound. This is illustrated in FIG. 2.

The wound dressing is made from a porous material that is permeable to air and liquid. It is preferably composed of textiles and can in particular have a thickness of 5 to 80 mm. The material can also be a sponge or another suitable material.

The cover film 1 is made from a material that is impermeable to air and liquid. It can be composed of a polymer. It preferably has a thickness of 0.1 to 5 mm.

The cover film 1 preferably has a shape memory. For this purpose, it can be made, for example, from a suitable polymer, in particular from block copolymers. Other suitable materials with shape memory are metals, for example Nitinol.

The shape memory means that the cover film 1 can change from a basic shape to a shape for use. This change preferably takes place at a normal human body temperature. The cover film 1 has the basic shape at a room temperature of about 20°. However, the change can also take place at room temperature or at a temperature of between 20° and 37°. In this case, the cover film must be stored cool and must be applied in the cooled state such that it has the basic shape when applied. Other varieties of shape-memory polymers can also be stimulated by magnetism or light.

The shape for use preferably contracts the wound and maintains the traction or pressure during drainage. This can be achieved, for example, by the basic shape being flat and the shape for use having a dome-shaped configuration in which the dome curves away from the wound. However, it is also possible that it simply draws together and thereby contracts the wound.

When using the above-described device, the wound dressing 2 is reduced to the size of the wound or of the wound bed and is adapted to the shape of the wound bed. It is then placed into the wound bed, and the cover film 1 is unfolded or rolled out and fixed on the surrounding skin. If the drainage tube 3 does not already protrude into the wound dressing 2, it is now pushed in and connected to a vacuum pump. A suitable vacuum for wound drainage can now be applied. The vacuum can be applied intermittently and with oscillations or in another known manner. The negative pressures known in the prior art are used. When the device is removed, the cover film and wound dressing can be withdrawn together or individually, depending on the design.

The device according to the invention can be easily adapted to the size and shape of the wound and is easy to handle.

### LIST OF REFERENCE SIGNS

1 cover film

2 wound dressing

3 drainage tube

C cavity

1. A device for treating wounds of a patient by means of low pressure, wherein the device comprises a wound dressing and a cover film, wherein the cover film is arranged on the wound dressing and connected thereto even before use of the device, wherein the cover film is at least partially folded or rolled up before use.

2. The device as claimed in claim 1, wherein a part of the cover film lies flat on the wound dressing before use.

3. The device as claimed in claim 1, wherein the cover film has a smaller surface area than the wound dressing when in the folded or rolled up state before use.

4. The device as claimed in claim 1, wherein the cover film is adhesively bonded on the wound dressing, welded to it or connected to it with a form fit.

5. The device as claimed in claim 1, wherein the device comprises at least one drainage tube that extends through the cover film and protrudes into the wound dressing, and wherein the cover film, in the area of this drainage tube, lies flat on the wound dressing.

6. The device as claimed in claim 1, wherein at least part of the rolled up or folded area of the cover film is self-adhesive.

7. The device as claimed in claim 1, wherein the cover film, in the state when unfolded or rolled out, has a greater surface area than the wound dressing and protrudes past this wound dressing about the entire circumference.

8. The device as claimed in claim 1, wherein the wound dressing is made from a textile.

9. The device as claimed in claim 1, wherein the cover film is made from polymer.

10. The device as claimed in claim 1, wherein the cover film has a shape memory and changes from a basic shape to a shape for use.

11. The device as claimed in claim 10, wherein the change to the shape for use takes place at a normal human body temperature.

12. The device as claimed in claim 10, wherein the shape for use contracts the wound.

13. The device as claimed in claim 10, wherein the shape for use has a dome-shaped configuration in which the dome curves away from the wound.

14. The device as claimed in claim 10, wherein the cover film has the basic shape at a temperature that corresponds approximately to room temperature.

15. The device as claimed in claim 10, wherein the cover film is made from a block copolymer.

16. The device as claimed in claim 1, wherein the cover film has a thickness of 0.1 to 5 mm.

17. A system for draining wounds, with a device for treating wounds as claimed in claim 1 and with a vacuum pump that can be connected to said device.

18. A method for using a device as claimed in claim 1, wherein the length and width of the wound dressing are adapted to a wound that is to be treated, by means of the wound dressing being made smaller, the wound dressing is placed into or onto the wound, and the cover film is unfolded or rolled out and is fixed on the skin surrounding the wound.
